(19) United States
(12) Patent Application Publication
(10) Pub. No.: US 2015/0208950 A1
(43) Pub. Date: Jul. 30, 2015

(54) Arterial and Venous Oxygenation Method and Apparatus

(71) Applicants: The Texas A&M University System, College Station, TX (US); Oak Ridge National Laboratory, Oak Ridge, TN (US); University of Pittsburgh - Of the Commonwealth System of Higher Education, Pittsburgh, PA (US); The United States Government, Washington, DC (US)

(72) Inventors: Tony J. Akl, College Station, TX (US); Gerard L. Coté, College Station, TX (US); Mark A. Wilson, Sewickley, PA (US); Milton Nance Ericson, Knoxville, TN (US); John P. Hanks, College Station, TX (US)

(21) Appl. No.: 14/608,145

(22) Filed: Jan. 28, 2015

Related U.S. Application Data
(60) Provisional application No. 61/932,567, filed on Jan. 28, 2014.
(60) Provisional application No. 61/932,575, filed on Jan. 28, 2014.

(51) Int. Cl. A61B 5/083; A61B 5/026; A61B 5/145; A61B 5/024; A61B 5/1455; A61B 5/1459

(52) U.S. Cl. CPC A61B 5/0833; A61B 5/14551; A61B 5/1459; A61B 5/14546; A61B 5/02416; A61B 5/026

(57) ABSTRACT

Methods and apparatuses for an oxygen consumption monitoring system are disclosed herein. In one embodiment, an oxygen consumption monitoring system is disclosed. The oxygen consumption monitoring system may comprise a probe, wherein the probe comprises a light source and a photodetector; and a main unit, wherein the main unit comprises a microcontroller and wireless transmitter. The probe may be hermetically sealed and may be capable of being implanted onto tissue. The photodetector may be capable of collecting reflectance data from the light emitted by the light source. The reflectance data may be capable of being sorted into arterial and venous blood oxygen consumption data for the tissue onto which the probe was placed or implanted. The data from the probe may be further sorted and processed to produce perfusion, heart rate, energy expenditure, caloric burn, blood pressure, hemoglobin concentration changes, and tissue oxidative stress.

### CROSS-REFERENCE TO RELATED APPLICATIONS

This application claims benefit of U.S. Provisional Application Ser. Nos. 61/932,567 entitled “Arterial and Venous Oxygenation Method and Apparatus” and 61/932,575 entitled “Non-Invasive Monitoring of Tissue Mechanical Properties”, both of which were filed on Jan. 28, 2014 and are incorporated herein by reference in their entirety.

This application is also related to a U.S. utility application filed concurrently herewith and entitled “Non-Invasive Monitoring of Tissue Mechanical Properties”, which is incorporated herein by reference in its entirety.

### STATEMENT REGARDING FEDERALLY SPONSORED RESEARCH OR DEVELOPMENT

This invention was made with government support under 5R01-GM077150 awarded by National Institutes of Health (NIH). The government has certain rights in the invention.

### BACKGROUND OF THE INVENTION

1. Field of the Invention

The present embodiments relate to measuring and separating venous and arterial oxygenation levels in vivo, and more specifically to measuring the oxygenation levels of tissue, heart rate, tissue perfusion, and total hemoglobin saturation to detect developing problems within the tissue or to ascertain the rate of tissue oxygen metabolism, energy expenditure, and caloric burn for either health or fitness applications.

2. Background of the Invention

Tissue oxygen metabolism as defined herein is the measure of oxygen consumption within a tissue. Traditionally, light has been used to measure arterial oxygen saturation and perfusion. Tissue is illuminated with different wavelengths of light. After light travels through the tissue, it is measured with a photodetector and has two major components: (1) an alternating current (AC); and (2) a direct current (DC). The alternating current pulsatile signal data is then related to the arterial oxygen level. Although the arterial oxygen level is useful for some applications, it does not provide the whole picture of tissue oxygen metabolism, specifically it does not quantify oxygen consumption and oxygen supply and demand and as such, may be too insensitive a measure for a clinician to provide timely intervention in the instance of a developing problem. Further, it does not provide the whole picture for health and fitness applications in terms of energy expenditure and caloric burn during periods of both rest and exercise, which is critical for health, as for example with obese patients, or for any fitness training or monitoring.

Current non-invasive technology such as pulse oximeters or near infrared spectroscopy focused solely on the AC signal may only provide a measure of arterial oxygenation or tissue oxygenation. As such, neither technology provides an accurate assessment of oxygen consumption and oxygen supply and demand. To address these failings, healthcare providers may use invasive methods such as blood catheters to access arterial and venous blood in order to measure the oxygen content. However, these invasive methods may create patient discomfort, may lead to vascular complications, and may also only provide data when a sample is directed to be taken. In such instances, the tissue oxygen metabolism state would then only be measured when requested by a physician. Relying on requested readings may expose the patient to increased risk of harm, should the doctor not provide sufficient oversight or deem repeated readings unnecessary. Further, such techniques are not conducive to health and fitness applications which require a noninvasive means of measurement.

As an example, in liver transplantation, the two weeks immediately post-surgery have the highest technical failure rate; however and despite knowing this, complications with the liver transplant are usually only detected after substantial damage to the graft has occurred. At this stage, usually the only option is a second transplant surgery which places the patient at risk and also wastes the first transplant, which perhaps may have been salvaged had the damage been detected sooner.

Another example involves the monitoring of tissue after the patient has suffered some degree of trauma. In a trauma situation the patient and consequently various patient tissue may accrue an “oxygen debt” due to excessive blood loss. In these situations, it may not be advisable to proceed with treatment until the tissue oxygen metabolism has stabilized. If the monitoring of tissue metabolism was more sensitive, treatments such as surgery may proceed sooner and therefore increase the chance of a positive outcome for some patients, conversely, a more sensitive measure of tissue oxygen metabolism may also provide better detection of instances where the tissue is still suffering from an oxygen debt and the risk of surgery is too high; thus reducing the risk of performing a premature operation.

In another example, the invention can be used to monitor heart rate, blood pressure, as well as oxygen consumption of tissue at rest or during exercise, which can be used to measure energy expenditure and caloric burn. This is useful for health and fitness applications such as, for example, in the case of obesity or any fitness training or monitoring.

Consequently, there is a need for a more sensitive quantification of tissue oxygen metabolism.

### BRIEF SUMMARY OF SOME OF THE PREFERRED EMBODIMENTS

These and other needs in the art are addressed in one embodiment by an oxygen consumption monitoring system comprising a probe and a main unit. The probe further comprises light sources and one or more photodetectors. The main unit drives the light sources, collects the data from the detectors, and then processes and displays the measurements. In some embodiments the main unit may transmit the data wirelessly to a processing and/or monitoring unit which may comprise a personal computing device (e.g., computer, smart-phone, tablet, and the like).

An additional embodiment comprises a method for measuring tissue oxygen metabolism in vivo using light sources, photodetectors, and data collection/manipulation. The method may comprise exposing tissue to light at different wavelengths generated by light emitting diodes, measuring the reflectance of the light via a photodetector to produce a reflectance signal. Analyzing and manipulating the reflectance signal such that an alternating and direct current signal are produced. Optionally, the method may further comprise reducing the alternating current and direct current to a measure of arterial and venous blood flow and then further relating the blood flow information to a measure of the oxygen metabolism of the tissue.

Another embodiment includes a probe and a main unit. The probe includes a light source and a photodetector. The main unit includes a microcontroller and a communications interface with the probe. The photodetector is configured to collect a reflectance data from a light emitted by the light source that illuminates a tissue or organ. The microcontroller processes the reflectance data into an arterial blood oxygen consumption data and a venous blood oxygen consumption data for the tissue or organ.

Yet another embodiment includes a method for determining a venous oxygenation and an arterial oxygenation of a tissue or an organ. A probe is provided that is affixed to or in close proximity to a surface of the tissue or organ, or subcutaneously inserted into the tissue or organ. The probe includes one or more light sources and one or more photodetectors. One or more processors communicably coupled to the probe and a data output device are also provided. The tissue or organ is illuminated using the one or more light sources, and a reflectance signal is detected using the one or more photodetectors. The venous oxygenation and the arterial oxygenation for the tissue or organ are determined based on the reflectance signal using the one or more processors. The venous oxygenation and the arterial oxygenation for the tissue or organ are then provided to the output device.

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other embodiments for carrying out the same purposes of the present invention. It should also be realized by those skilled in the art that such equivalent embodiments do not depart from the spirit and scope of the invention as set forth in the appended claims.

### BRIEF DESCRIPTION OF THE DRAWINGS

For a detailed description of the preferred embodiments of the invention, reference will now be made to the accompanying drawings in which:

FIG. 1A illustrates a schematic of the collected reflectance signal;

FIG. 1B illustrates a spectrum of the reflectance signal collected prior to amplification and with the DC signal omitted;

FIG. 2 illustrates a flowchart of the general process;

FIG. 3 illustrates a flowchart in which the values obtained from the oxygen consumption model system may also be used in conjunction with other measurements to determine values for other metrics of interest;

FIG. 4 illustrates heart rate changes as measured by the arterial pressure catheter and the telemetry sensor;

FIG. 5A illustrates hemoglobin oxygenation index (right axis) measured by the optical telemetry system versus venous oxygen saturation and hepatic supply oxygen saturation (left axis);

FIG. 5B illustrates hemoglobin oxygenation index (right axis) measured by the optical telemetry system versus venous and hepatic supply oxygen saturation (left axis);

FIG. 6A illustrates venous oxygen saturation as measured by the telemetry sensor and the central venous catheter;

FIG. 6B illustrates venous oxygen saturation as measured by the telemetry sensor and the central venous catheter;

FIG. 7 illustrates a scatter plot of the predicted (telemetry) versus measured (catheter) venous oxygen saturation for FIGS. 6A and 6B;

FIG. 8A illustrates a mixed oxygen supply (MOS) measured by the telemetry sensor and the reference equipment, where MOS is a measure of hepatic supply oxygen saturation;

FIG. 8B illustrates a mixed oxygen supply (MOS) measured by the telemetry sensor and the reference equipment;

FIG. 9 illustrates a scatter plot of the predicted vs. measured MOS for FIGS. 8A and 8B;

FIG. 10A illustrates a predicted venous oxygen saturation by combining the DC NIRS measurements with the AC pulse oximetry measurements;

FIG. 10B illustrates a predicted venous oxygen saturation by combining the DC NIRS measurements with the AC pulse oximetry measurements;

FIG. 11A illustrates total hemoglobin concentration (ΔHbT) in hepatic tissue measured by the telemetry sensor versus total hepatic flow measured by the addition of the HA and PV Transit-Time flowmeters' measurements;

FIG. 11B illustrates total hepatic flow and mean arterial pressure and shows that the increase in flow after t=200 min is accompanied by an increase in the arterial pressure suggesting that a systemic response is responsible for that increase;

FIG. 12A illustrates a total hemoglobin concentration (ΔHbT) and flow changes; and

FIG. 12B illustrates a scatter plot of measured hemoglobin concentration change (ΔHbT) vs. tissue perfusion (flow normalized by liver weight).

### DETAILED DESCRIPTION OF THE PREFERRED EMBODIMENTS

Embodiments may comprise one or more probes. The probes may be placed at a specific application and measurement site. The probes may be composed of any material sufficient for contact with the internal or external structures of a living organism. Such materials may be defined as biocompatible materials. Examples of general materials include, but should not be limited to metals, plastics, and the like. Without limitation, specific examples of materials may include polyethylene glycol, poly(methyl methacrylate), polydimethylsiloxane, parylene, titanium, combinations and composites thereof, and the like. Alternatively, the probes may be encapsulated using any of the above mentioned materials such that any portion of the probes (e.g., the electronic portions of the probes) is hermetically sealed and/or moisture tight.

Embodiments of the probes may further comprise one or more light sources. Multiple light sources may be used for a given application, including a combination of different models or types of light sources. The light source may be any light source sufficient for measuring the levels of tissue oxygen metabolism. Examples include light emitting diodes (LEDs), lasers, etc. The light source may produce light of any wavelength. Light sources that produce wavelengths of light in the near infrared range penetrate deeper in the tissue and may carry higher perfusion and oxygenation signal levels and thus may be preferred for some applications. Examples of potential light wavelengths include approximately 735, 805, and 940 nm. Optionally, the LEDs may be time multiplexed or frequency multiplexed such that only a single photodetector may be needed to collect the diffuse reflectance at each of the wavelengths. Moreover, the light produced from the light source may be modulated such that the produced light is at a different frequency than the ambient light and may therefore be distinguished from any ambient light noise. Modulation may comprise frequency modulation, time division multiplexing, or a combination of the two. Any technique for modulation that allows the light source to produce a light at a different frequency than the ambient light of the surrounding system may be sufficient for applications.

Embodiments of the probe may further comprise one or more photodetectors. In embodiments where multiple photodetectors are used, the photodetectors may be set up as an array. The photodetector may be any photodetector sufficient for measuring the light reflectance of the light source. Without limitation, the photodetectors may comprise solid state photodetectors, specific examples of which may include silicon photodetector, photo multiplier tubes, charge-coupled devices (CCD), avalanche photodiodes, electron-multiplying charge-coupled device, and the like. Certain types of photodetectors, such as CCD photodetectors may comprise cameras. Multiple photodetectors may be used for a given application, including a combination of different models or types of photodetectors. The photodetector should be sensitive to the wavelength of light produced by the light source. In some embodiments a single photodetector may measure the reflected light from multiple light sources. The photo detector may be composed of any material sufficient for measuring reflected light and potentially also sufficient for contact with the internal structures of a living organism. Examples of materials include metals, plastics, and the like.

In embodiments, the probes may be used invasively or noninvasively. For example, the probes may be implantable such that is affixed to the tissue or organ either on the surface of the tissue or organ or subcutaneously inserted into the tissue or the organ. The probes may be affixed to or inserted into the tissue of the organ or the organ itself in any manner sufficient for the specific desired application. In alternative embodiments, the probes may reside on the surface of a body. The probe may be affixed to the surface of the body such that it resides next to and in close proximity to the skin of the body. The probe may be affixed to the surface of the body in any means sufficient for a specific desired application. Such means may include bands, wrappings, stickers, tape, adhesive materials/solutions, and the like. The probe may be affixed to any part or portion of the body such as internal organs, limbs, hands, fingers, core, torso, head, neck, etc.

In further alternative embodiments, the probe may be a handheld device. The handheld probe device may comprise any of the light sources or photodetectors described above and in any combination as described above. Preferred embodiments of the handheld probe comprise a wide field camera photodetector (e.g., a CCD photodetector). In embodiments of the handheld probe comprising an array of photodetectors, the handheld probe may take other body measurements such as blood pressure.

In embodiments, the probes may monitor the vessels supplying blood to various organs and organ systems. For example, in a specific embodiment, three probes are positioned to measure oxygen metabolism of the liver tissue. One probe may be positioned to measure the hepatic artery, another to measure the portal vein, and a third to measure hepatic parenchymal tissue. In this embodiment, it may not be necessary to measure any tissue except for the hepatic parenchymal tissue, however the additional probes may be used as references. In embodiments, the probe may be affixed to the tissue using any sufficient means. In alternative embodiments, the probe may be located on a handheld apparatus and positioned over and/or focused on the tissue to be examined. In further embodiments, the probe may be placed on or into the tissue such that it may remain for a desired measure of time until monitoring is no longer needed or it is desirable to remove it.

Embodiments may comprise a main unit. In some embodiments, the main unit may drive the light sources, collect the data from the detectors, and/or process and display the collected data as measurements. The main unit may be a component of or may be separate from the probe. In embodiments where the main unit is separate from the probe, the main unit may connect wirelessly with the probe; in further alternative embodiments, the probe may dock and/or mate with the main unit such that the probe and the main unit may interact. Without limitation, examples of docking and/or mating may include use of wire interface (e.g., USB, Ethernet, serial interface, and the like). In alternative embodiments, the main unit may be tethered to the probe such that it is connected to the probe yet at a distance away from the tissue or body to be examined. The main unit may comprise one or more circuit boards. The main unit may comprise one or more microcontrollers. The main unit may communicate wirelessly with a remote relay station and/or a remote personal computer such as a computer, smart-phone, tablet, etc. In embodiments wherein the main unit is a component of the probe, the main unit may be encapsulated in the same manner as any other component of the probe may be encapsulated. In embodiments, wherein the main unit is distinct from the probe, the main unit may be encapsulated or may not be encapsulated.

Embodiments may comprise a transmitter and/or a receiver for wireless communication. The transmitter and/or receiver may be a component(s) of the probe and/or the main unit, either individually or in conjunction with each other. In embodiments, the system may communicate with any mobile device (i.e., phone, smart watch, etc.) directly or through a relay unit. Without limitation, the transmitter and receiver may comprise communication means such as radio waves, infrared signals, audio, and electro-magnetic waves, for example active RF (e.g., WiFi, WiFi 802.11, Bluetooth®, 3G, and the like), RFID (e.g., Near Field Communication (NFC), both active and passive RFID as well as low and high frequency and the like), an infrared or optical link (LED's and the like), and/or any other suitable data transfer type, device, or method.

Embodiments may comprise a power source. The power source may be any type of battery (primary or secondary) capable of providing power to drive the main unit and the probe for the desired data collection duration. Specific examples of batteries include but are not limited to lithium ion batteries, lithium/carbon monoflouride (Li/CFx), lithium/silver vanadium oxide (SVO), lithium iodine, alkaline batteries, nickel-zinc batteries, or other battery technologies. The power can also be supplied through an alternative source or sources including inductive power coupling, optical, ultrasonic/ultrasound, motion, or a scavenged energy source (heat, vibration, ambient light, chemical, or acoustic). Depending on the requirements of the application, a combination of these methods may be used such as lithium ion batteries charged via inductive power coupling.

Embodiments may comprise a method for measuring perfusion, heart rate, oxygen consumption and oxygen supply and demand and then calculating the tissue oxygen metabolism, energy expenditure, or caloric burn. The method comprises illuminating tissue with different wavelengths of light and collecting the reflected light data with a photodetector after the light has propagated through the tissue. The reflected light data collected by the detector may then comprise a pulsatile alternating current component that may be used to measure the arterial blood oxygenation. The remainder of the reflected light data may comprise a direct current that comprises the non-pulsating arterial blood oxygenation level and the venous blood oxygenation level. The method further comprises manipulating the data such that the venous component of the tissue oxygenation measurement may be calculated such that and because the arterial component of the tissue oxygenation measurement is now known.

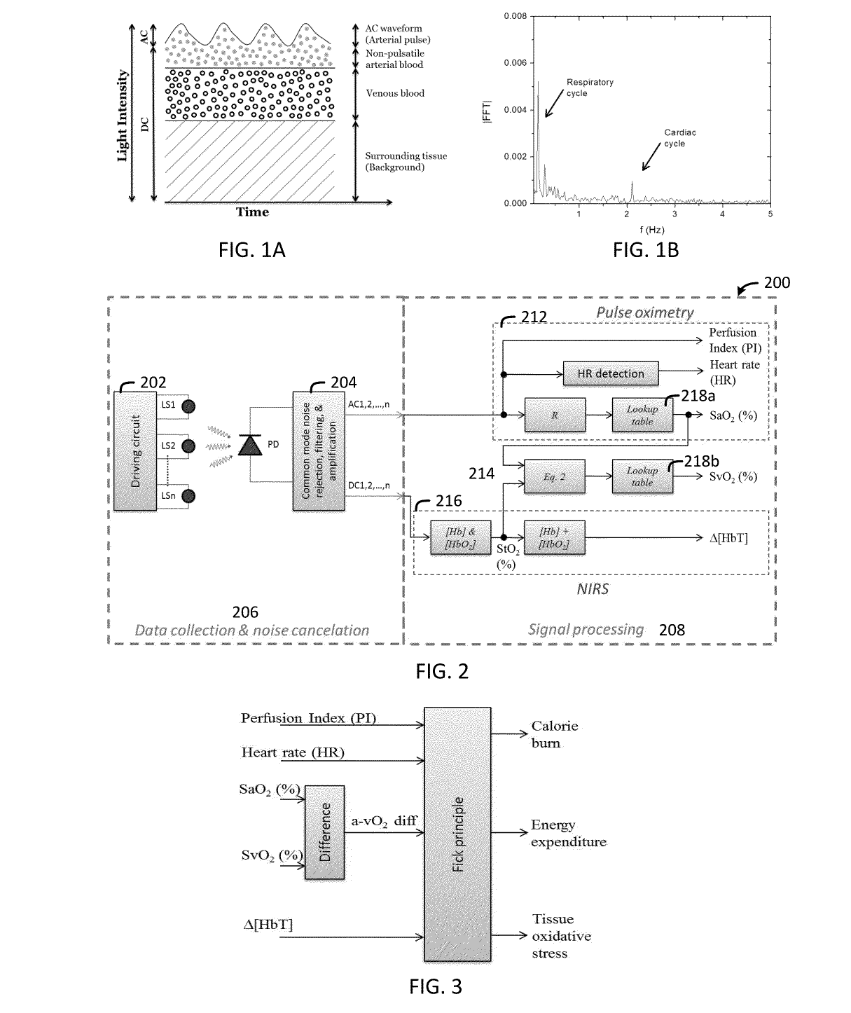

In a specific embodiment and as an example, the pulsatile nature of the arterial blood flow causes periodic fluctuations in the blood volume contained in the probed tissue, the collected signal therefore has a pulsatile component transduced as an alternating current by the photodetector and can be used to assess changes in the arterial blood supplying the tissue with oxygen and nutrients. This pulsatile component is typically weak (0.02% to 20%) and may in some embodiments require amplification. This waveform constitutes a photoplethysmogram. FIG. 1A shows a schematic of the different components in the collected signal, which includes an AC component (arterial pulse) and a DC component (non-pulsatile atrerial blood, venous blood and surrounding tissue (background)). In addition to the cardiac cycle pulsations in the AC component, there are other low frequency waveforms that can be detected in the reflectance signal such as the respiratory cycle and some vascular auto-regulation processes. The remainder of the collected signal is a non-pulsatile direct current component carrying information about surrounding tissue and the resting blood volume which has a venous and arterial portion. FIG. 1B shows the spectrum of a typical reflectance signal collected in vivo with the system described herein prior to the alternating current amplification. Note that since the direct current component is much higher than all the alternating current components it has been removed in this graph to better visualize the alternating current peaks as a function of frequency. Separating these two quantities (AC and DC) allows for the quantification of tissue oxygen consumption. This can be used to study the metabolic activity and relate it to tissue stress. Low arterial oxygen levels indicate a deficiency in oxygen supply to the tissue. On the other hand, low levels of venous oxygenation indicate a high consumption rate (low perfusion levels) due to extraction of oxygen by the tissue. These two levels coupled with a relative perfusion measurement can be used to detect hemodynamic complications and identify potential causes. Various embodiments described herein simultaneously measure venous oxygen saturation, arterial oxygen saturation, and perfusion levels to provide physicians with a more complete picture of graft hemodynamics in order to assess graft function intra- and post-operatively.

In embodiments, the separated quantities for the alternating current and the direct current provide the quantification of tissue oxygen metabolism. This quantification may then be used as a diagnostic for the physician to measure tissue stress. For example, low arterial oxygen levels indicate a deficiency in oxygen supply to the tissue. On the other hand, low levels of venous oxygenation indicate a high consumption rate (low perfusion levels) due to extraction of oxygen by the tissue. The physician, therefore, may then be able to better direct treatment options for the patient.

In embodiments, the oxygen consumption monitoring system may also be used to monitor metrics other than and/or in addition to oxygen consumption. Such metrics may be extrapolated from the oxygen consumption monitoring methods described above or may be extracted from the AC and DC signals collected by the oxygen consumption monitoring system. For example, the oxygen consumption monitoring system may monitor perfusion, caloric burn, heart rate, blood pressure, energy expenditures, hemoglobin concentration change, and the like. This information may be used by itself or in conjunction with the oxygen consumption measurements or other metrics to produce diagnostic information for healthcare providers or information relevant to the state of the tissue that may be used directly by the patient themselves for fitness applications and the like.

In embodiments, a calibration model may be used to calculate venous oxygenation from the tissue oxygenation and arterial oxygenation levels. In embodiments, the consumption monitoring system may comprise a display or monitor such that information about arterial oxygen saturation, venous oxygen saturation, tissue oxygen saturation, oxygen consumption rate, oxygen extraction rate, heart rate, and/or respiratory rate, etc. may be displayed in such a manner to easily convey the details of this data to a mobile platform, a monitoring physician or other type of healthcare provider.

Various non-limiting examples of embodiments of the present invention will now be described. The oxygen consumption monitoring system developed for these studies consists of three sensors. Two of the sensors were equipped with vascular probes to monitor the vessels supplying blood to the liver, the hepatic artery (HA) and the portal vein (PV). The third probe was used to monitor changes in the hepatic parenchymal tissue. The HA and PV probes served as reference measurements and are not required for the final application since the measurements and calculations can be performed with the parenchymal probe alone. The system consisted of four printed circuit boards, three of which are identical sensor interface boards each having the full functionality of a sensor including three programmable amplitude, time-multiplexed LED drives, and synchronized detector signal amplification, dark current subtraction, filtering, and digitization. The fourth board included the microcontroller unit that communicates with and controls the three sensors via the sensor interface boards. The microcontroller also communicates wirelessly with a relay unit connected to a laptop computer. The computer sends the acquisition parameters, provides the visual interface, and allows further data analysis and storage. The computer graphical user interface was developed in Python to control the sensors, and allow the user to visualize the data in real time. All electronics and a rechargeable lithium-ion battery (BatterySpace.com, part# CU-MM184) were encapsulated in a custom plastic box (90.17×80.00×62.23 mm3) built using a 3D printer and coated with polydimethylsiloxane (PDMS). The three probes were constructed using multi-wavelength LEDs (Epitex L660/735/805/940-40B42) and a silicone photodetector (Hamamatsu S2833-01) soldered to a custom printed circuit board. Note that these LEDs contain four wavelengths in a single package; however only three wavelengths (735, 805, and 940 nm) were used in the sensors. The probes were also coated with PDMS to avoid any leakage to the electronics. The source to detector separation was set to 4 mm edge to edge (˜8.7 mm center to center).

Each of the sensors extracts an AC signal from the collected diffuse reflectance (band-pass filter f3 dB˜0.7 and 24.7 Hz) and amplifies it by a gain specified by the user (1 to 96 times). Both the DC and AC signals are transmitted to the data acquisition relay unit and saved on a computer for further processing.

Fourier processing was used to measure the amplitude of the pulsatile wave. The Fast Fourier Transform (FFT) was calculated for 25 s data intervals using software developed in MATLAB (Mathworks, Inc.). The software detects the frequency peak that corresponds to the cardiac cycle and uses it as the amplitude of the AC signal. The frequency of that peak was used to measure the heart rate in beats per minute (60*fₚₑₐₖ).

Using the AC amplitude at the 735 and 940 nm wavelengths, the modulation ratio R that is typically used in pulse oximeters to assess oxygen saturation was calculated (see Equation 1a below). This quantity requires a calibration curve to produce quantitative oxygen saturation results. To calibrate the sensor the data was fitted to a calibration model in the form shown in Equation 1b.

$$\begin{matrix}R = \frac{{AC}_{735} / {DC}_{735}}{{AC}_{940} / {DC}_{940}} & ( 1 a ) \\ {SO}_{2} = \frac{a . R + b}{c . R + d} & ( 1 b )\end{matrix}$$

The quantity R is a measure of the ratio of absorbance at the red and NIR wavelengths of the pulsatile perfusion. Due to the compliance of the tissue, blood flow loses its pulsation on the venous side. Thus R is used to follow the changes in the oxygen supply (i.e., arteries and the arterial side of the capillary network).

The DC signals were processed using the equations employed for Near Infrared Spectroscopy (NIRS) signals as shown below. The pathlength factor (PF) was estimated using the theoretical equation from the optical properties of the tissue under investigation. These equations are used to derive the change in concentration of oxyhemoglobin, deoxyhemoglobin, and total hemoglobin (ΔHbO₂, ΔHb, and ΔHbT respectively). The change in total hemoglobin concentration (ΔHbT) tracks perfusion. To obtain a measure of the change in oxygenation, the hemoglobin oxygenation index (ΔHbD=ΔHbO₂−ΔHb) was used. During perfusion changes, ΔHbO₂ and ΔHb vary similarly and the difference (ΔHbD) remains unaltered. However, for oxygenation changes, ΔHbO₂ and ΔHb vary in opposite directions leading to a change in the hemoglobin oxygenation index (ΔHbD). The equations used for the calculation of the pathlength factor are presented below:

$$Δ Hb = \frac{{ɛ}_{HbO 2}^{λ 2} \frac{Δ {OD}^{λ 1}}{{PF}^{λ 1}} - {ɛ}_{HbO 2}^{λ 1} \frac{Δ {OD}^{λ 2}}{{PF}^{λ 2}}}{( {ɛ}_{Hb}^{λ 1} {ɛ}_{HbO 2}^{λ 2} - {ɛ}_{Hb}^{λ 2} {ɛ}_{HbO 2}^{λ 1} ) L}$$

$$and$$

$$Δ HbO 2 = \frac{{ɛ}_{Hb}^{λ 1} \frac{Δ {OD}^{λ 2}}{{PF}^{λ 2}} - {ɛ}_{Hb}^{λ 2} \frac{Δ {OD}^{λ 1}}{{PF}^{λ 1}}}{( {ɛ}_{Hb}^{λ 1} {ɛ}_{HbO 2}^{λ 2} - {ɛ}_{Hb}^{λ 2} {ɛ}_{HbO 2}^{λ 1} ) L}$$

$$and$$

$$Δ HbT = Δ Hb + Δ HbO 2$$

where the pathlength factor is given by the equation:

$$PF = \frac{1}{2} {( \frac{3 {μ}_{s}^{′}}{{μ}_{a}} )}^{1 / 2} [ 1 - \frac{1}{( 1 + {L ( 3 {μ}_{s}^{′} {μ}_{a} )}^{1 / 2} )} ]$$

and
OD is the measured optical density from the DC level.

The values calculated using the DC levels as described above represent tissue oxygenation which may be affected by either changes in oxygen supply (arterial side) or in oxygen consumption (venous side). It is of paramount importance to separate the two contributions to be able to possibly diagnose the cause of complications when they occur. This ratio is different for various types of tissue and probe geometries. If this ratio is determined it can be used to extract the venous signal contribution to the DC levels. To accomplish that Multiple Linear Regression (MLR) was used, and the measured venous oxygenation was fit to the linear combination of the DC measured changes (ΔHbD) and the oxygen saturation of the supply (Equation 2).

SvO₂=a·DC+b·SO₂+c (2).

Equation 2 is a multiple regression equation. For this specific example it is a linear equation, however, some models and/or applications may require a non-linear multiple regression.

A flowchart of the general process 200 is shown in FIG. 2. The probes 202 and the main unit 204 are represented by the electronics on the left side (Data collection & noise cancellation 206) whereas the data analysis of the collected signal is represented on the right side (Signal processing 208). The probe 202 can be invasive or non-invasive. For example, the probe 202 can be affixed to or in close proximity to a surface of the tissue or organ, or subcutaneously inserted into the tissue or organ. Moreover, the probe 202 can be integrated into, directly connected, tethered or wirelessly connected to the main unit 204. Other possible characteristics and configurations of the probes 202 and main unit 204 were previously described.

The driving circuit 202 includes one or more light sources (e.g., LS1, LS2 . . . LSn) that illuminate the tissue or organ with a light having one or more wavelengths (e.g., 735, 805, 940 nm, etc.). The main unit 204 provides common mode noise rejection, filtering and application of the reflectance signal 210 received by the one or more photodetectors (PD). The reflectance signal 210 includes an AC component (AC1, 2, . . . , n) and a DC component (DC1, 2, . . . , n) (See e.g., FIGS. 1A and 1B). The signal processing 208 can be performed using one or more processors within the main unit 204 or remotely located with respect to the main unit 204. In this example, the signal processing 208 includes pulse oximetry processing 212, venous oxygenation processing 214 and NIRS processing 216. The signal processing 208 determines an arterial oxygenation (SaO₂) based on the AC component (AC1, 2, . . . , n) and a venous oxygenation (SvO₂) for the tissue or organ based on both the AC component (AC1, 2, . . . , n) and DC component (DC1, 2, . . . , n) of the reflectance signal 210. The lookup table 218 is a conversion chart wherein the value of R is described in terms of oxygen saturation for arterial blood flow (218a), and the values obtained from Equation 2 are described in terms of oxygen saturation for venous blood flow (218b). The signal processing 208 can also provide other data, such as perfusion index (PI) and heart rate (HR) from the AC component (AC1, 2, . . . , n) of the reflectance signal 210, and change in total hemoglobin concentration (ΔHbT) from the DC component (DC1, 2, . . . , n) of the reflectance signal 210, etc.

The values obtained from the oxygen consumption model system may also be used in conjunction with other measurements to determine values for other metrics of interest. This process is illustrated in the flowchart of FIG. 3, wherein the difference between arterial and venous oxygen saturation as computed above may be used in conjunction with the perfusion index, heart rate, and/or the total change in blood hemoglobin to calculate caloric burn, energy expenditure, and/or tissue oxidative stress.

An animal study using the oxygen consumption monitoring system was performed on two swine (a 21 kg male and a 27 kg female). Prior to anesthesia, the animals were premedicated with Telazol (5-10 mg/kg intramuscularly, im) and the analgesic Buprenorphine (0.01-0.05 mg/kg, im). To induce anesthesia, the animals were administered 3-4% Isoflurane in oxygen at 3 L/min via a face mask. An endotracheal tube was inserted into the trachea, secured in place and connected to a mechanical ventilator (8-12 BPM and tidal volume of 5-10 mL/lb). Isoflurane (0.5-4%) was administered in oxygen to maintain anesthesia. A laparotomy was performed to expose the liver and its vasculature. Transit-time ultrasound probes were placed on the hepatic artery (Transonic Systems, cat# MA4PSB) and the portal vein (Transonic Systems, cat# MA10PSB) to monitor flow changes. An oxygenation catheter was placed in the aorta via the iliac artery (Edwards Lifesciences, cat# XA3820HKCDC) and connected to a Vigilance Monitor (Edwards Lifesciences). Another oxygenation catheter was placed in the inferior vena cava at the level of the hepatic veins to monitor venous oxygenation changes. Two laser Doppler flowmeter probes were placed on the parenchyma to monitor tissue perfusion changes. A catheter was placed in the femoral artery to monitor arterial pressure. Throughout the experiments, vital signs (body temperature, SpO₂, heart rate, blood pressure, etc.) were monitored closely and recorded. All reference equipment was connected to a custom built data acquisition system and saved for further processing. Similarly, three probes from the optical telemetry system were placed on the HA, PV, and the liver parenchyma. All probes were secured using 5/0 polypropylene sutures. Note that the HA and PV probes were placed for reference; however, no vascular probes will be required for the final application. Vascular occluders (Harvard Apparatus, part.# PY2 62-0111, -0113, and -0117) were placed on the HA and PV to be able to alter hepatic flow and perfusion. Hypoxia was induced by inhalation of low oxygen content mixtures. The wireless sensor electronic unit was placed outside the body next to the animal on the surgical table and tethered to the probes. The relay unit was connected to a laptop placed approximately 4 feet from the surgical table. Data were collected intermittently prior, during, and after any flow or oxygenation perturbation. At the end of the experiment, while still anesthetized, the animal was euthanized with a barbiturate derivative solution administered intravenously (80-120 mg/kg). Finally, livers were extracted and weighed (465 and 525 g for the 21 and 27 kg animals respectively) to convert the flow measurements (mL/min) into an average tissue perfusion measure (mL/min/g of tissue). All studies were performed under an animal use protocol (AUP #2010-257) approved by the Institutional Animal Care and Use Committee at Texas A&M University.

The liver has a complex vasculature that is supplied by two different vessels: the hepatic artery (HA) and the portal vein (PV). The HA supplies approximately 25% of nutrients and total blood flow but it is rich in oxygen and delivers approximately 75% of the total liver oxygen supply. The PV is part of the venous system but supplies blood to the liver. The portal venous blood is rich in nutrients but relatively poor in oxygen, and it supplies roughly 75% of liver nutrients along with 25% of its oxygen. Thus, the supply oxygenation in the liver tissue is not that of the artery alone, but it is the addition of the contribution of the PV and the HA. Hereinafter, this quantity will be referred to as the mixed oxygen saturation (MOS) as defined in Equation 3. Due to the limited exposed space on the PV (few centimeters) and the required flow probes and vascular occluders placed on that vessel, a catheter could not be inserted into the PV to monitor its oxygenation. It was assumed to be equal to the non-hepatic venous oxygenation for the purpose of the calculations.

$$MOS ( \% ) = \frac{{Flow}_{HA} . {SaO}_{2} + {Flow}_{PV} . {SvO}_{2}}{{Flow}_{total}} \qquad ( 3 )$$

Note that MOS represents the oxygen supply to the liver and will be compared to the oxygen saturation predicted by the pulsatile AC signal as discussed earlier. Similarly, total hepatic flow is the sum of PV flow and HA flow. In addition to the Laser Doppler Perfusion monitor, the total flow was used as measured by two Transit-Time vascular flow monitors to track tissue perfusion as described by Equation 4 below:

Flowₜₒₜₐₗ=Flow_{HA}+Flow_{PV}αTissue Perfusion (4)

Two different data collection procedures were used on the two animals. For the first study, hypoxia was induced without imposing any change in the hepatic flow to test the ability of the sensor to track oxygenation changes. The second study began with four consecutive hepatic artery occlusions, followed by three portal vein occlusions. These occlusions were performed at normal systemic oxygenation levels. Hypoxia was induced afterwards, and vascular occlusions (HA and PV) were performed again at low systemic oxygenation levels. All occlusions were brief (less than 1 minute for full occlusion) and were performed in gradual steps. Although the inhaled oxygen level was not changed during occlusions, the hepatic oxygen saturation is expected to be altered due to an increased hepatic oxygen extraction ratio and a change in the relative flow (see the MOS equation above) of the HA (high oxygen content) and PV (low oxygen content).

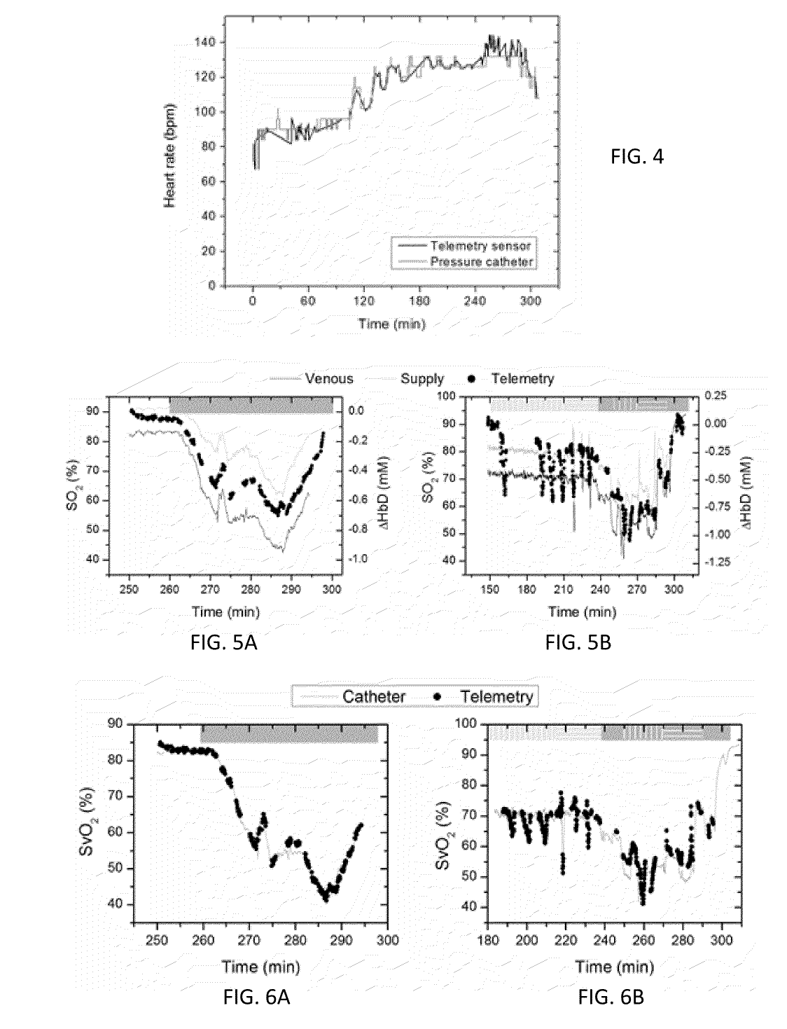

To verify that the pulsatile signal is tracking the cardiac cycle, the cardiac cycle peak detected by the system was looked at and compared to the heart rate measured by the arterial pressure catheter. This was performed on data from both animals and the detected heart rate was accurate with a Root Mean Square Error (RMSE) of 3.9 bpm (0.065 Hz). Some of this error is due to the difference in the integration time between the telemetry sensor (25 s) and the pressure catheter (2 s). FIG. 4 shows the heart rate throughout the study as measured from the arterial pressure catheter and the FFT of the photoplethysmography (PPG) signal measured with the telemetry sensor.

To track oxygenation changes, the hemoglobin oxygenation index (ΔHbD) was computed, as obtained from the measured DC levels, which, as discussed above, is a measure of tissue oxygenation affected by both arterial and venous oxygen saturation levels. This level is compared to both oxygen supply (MOS) and venous oxygenation (SvO₂) from the two different studies as shown in FIGS. 5A and 5B. As described earlier, for FIG. 5A hypoxia was induced and perfusion was not altered. However, in the second study, FIG. 5B, multiple occlusions of the HA and PV at various levels of oxygen saturation occurred during hypoxia. Vascular occlusions have been shown to alter tissue perfusion and oxygenation in liver tissue. In the following graphs, hypoxia periods are indicated by a dark grey box on the upper horizontal axis. Note that the recovery period from hypoxia is also included in the grey boxed region. Vertical white lines indicate segments where one or more hepatic artery occlusions were performed while horizontal white lines indicate where one or more portal vein occlusions were performed. The occlusion periods include multiple occlusions and baseline readings in between.

FIGS. 5A and 5B indicate that the measured hemoglobin oxygenation index is tracking oxygenation changes. This measure was obtained from the collected DC signal that is probing both the HA/PV supply and the post-hepatic venous components. To verify that this measure contains oxygenation information about both supply and hepatic venous blood, the measurements were correlated to the reference data obtained from the oxygenation catheters and flow meters. Although ΔHbD correlated well with both supply and venous oxygenation (data shown in supplementary information), it correlated best with a combination of the two using multiple linear regression (MLR) analysis described by Equation 5. The coefficient of determination (R²) was 0.99 for study 1 (no vascular occlusions) and 0.80 for FIG. 5B (vascular occlusions at multiple levels of oxygenation) respectively. This corresponds to a root mean square error (RMSE) of 1.39% and 3.93% respectively.

ΔHbD=a·MOS+b·SvO₂+c (5)

Having the calibration coefficients (a, b, and c) from the MLR, the mixed oxygen saturation (MOS) measured by the gold standards, and the hemoglobin oxygenation index (ΔHbD) measured by the telemetry system, Equation 5 was used to compute venous oxygen saturation (SvO₂). FIGS. 6A and 6B below show the predicted venous oxygenation and the measured oxygenation by the venous catheter as a function of time. Note that in FIG. 6B on the right, occlusions are performed on the hepatic artery and are always seen as a decrease in the measured venous oxygenation by the probe. However, this decrease is not detected by the venous catheter during the first three occlusions because the catheter was measuring hemoglobin oxygen saturation in the vena cava rather than the hepatic veins. Thus, the first three occlusions performed on the hepatic artery caused a decrease in the hepatic venous oxygen saturation as shown by the probe but did not have a substantial effect on the systemic venous oxygenation, where the venous catheter is measuring, since the HA flow is relatively low (7% of total cardiac output) compared to the portal vein flow (22% of total cardiac output).

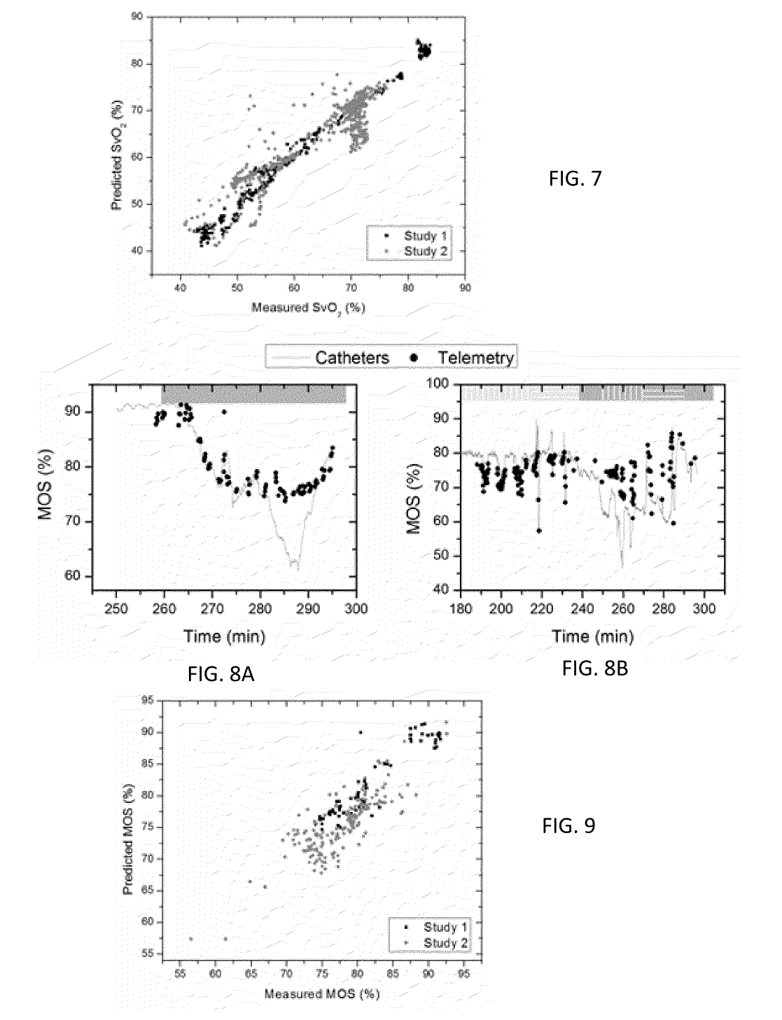

The data from FIGS. 6A and 6B are shown on FIG. 7 as a scatter plot. Note that the data from Study 2, FIG. 6B, has a higher RMSE (3.93%) compared to Study 1, FIG. 6A. (1.39%). This increase is mainly due to the occlusions performed in that study, and part of this perceived error is from the reference measurements, and not the system, because it was probing central venous oxygenation and not the hepatic vein.

The data shown in FIGS. 6A, 6B, and 7 use the supply oxygen saturation levels obtained from the catheters to extract the venous oxygenation from the DC levels. However, as described earlier, it is desirable to determine the supply oxygenation levels from the pulsatile signal and avoid using any additional reference measurements. To do so, the modulation ratio (R) was calculated, and measurements were calibrated as discussed in the materials and methods section. The data from each experiment were calibrated separately. These measurements were compared to the Mixed Oxygen Saturation (MOS) described earlier. FIGS. 8A and 8B shows the measured MOS and the reference data from the catheters and the flowmeters computed using Equation 3. The modulation ratio was able to predict the MOS except when the oxygen saturation dropped below 75%. This is a known problem of pulse oximeters and is mainly due to the high attenuation of the red wavelength (735 nm) for low oxygenation levels. If needed, this issue can be resolved by optimizing the sensor design (illumination wavelength, amplification, etc.) to operate for low oxygen saturation levels. During study 2, FIG. 8B, the oxygen saturation during vascular occlusions dropped to as low as 56%, and the sensor was still able to measure it accurately. It is believed that this is due to the decreased absorbance as a result of the perfusion decrease that allowed the red wavelength (735 nm) to still be measured accurately.

For oxygenation levels above 72% (for normal perfusion levels) and 55% (for low perfusion levels) the calculated modulation ratio from the pulsatile signal was able to predict oxygenation changes with an RMSE of 2.19% and 2.82% for study 1 and 2 respectively. FIG. 9 shows the corresponding scatter plot.

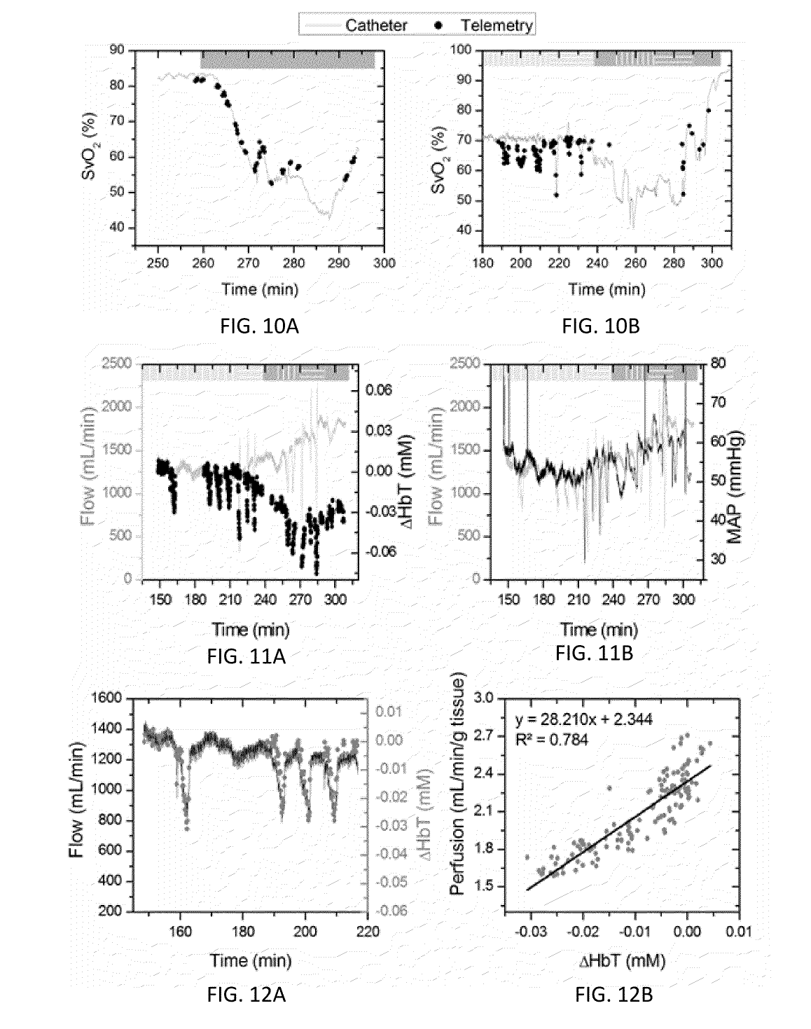

These predicted MOS levels can be used with Equation 5 to predict venous oxygenation SvO₂ without the need for a reference measurement. Because the pulsatile signal could not predict data for very low oxygen saturations, this concept was tested on all other parts of the data, and the measurements that were obtained are shown in FIGS. 10A and 10B. For study 1, FIG. 10A, venous oxygen saturation was measured with an RMSE of 1.17% (R²=0.986) while for study 2, FIG. 10B, the RMSE was higher at 3.44% (R²=0.1). This increased prediction error is mainly due to the changes measured during HA occlusions that are not reflected in the central venous oximetry catheter measurements. However, it is believed that these changes likely reflect variations in the hepatic venous oxygenation.

One of the important features of the presented sensor is the ability to track perfusion and oxygenation changes simultaneously. To track perfusion changes, the change in tissue total hemoglobin concentration (ΔHbT) was measured as described above. After analyzing the laser Doppler flowmetry (LDF) data from the commercial system, the LDF signal was found to be correlated with changes in HA flow but not PV nor total flow (R-square=0.8, 0.1, and 0.5 respectively) measured by vascular transit-time ultrasonic flowmeters. It is believed that the LDF was probing a branch of the HA and was not tracking tissue perfusion. Because the LDF measurements reflected the HA flow and not tissue perfusion, they were not used as a reference for parenchymal perfusion. Instead, the total hepatic flow measured by the addition of the signal from two transit-time ultrasound flowmeters (HA & PV) was used as the reference for parenchymal perfusion measurements.

FIG. 11A shows the changes in hemoglobin concentration measured by the optical telemetry sensor and the total hepatic flow measured by the transit-time ultrasonic flowmeters. The two quantities correlate with high accuracy in the first 70 minutes of the study (t=150-220 min) during which four HA occlusions were performed. After the first PV occlusion (t=220 min), the signal from the optical telemetry sensor showed a slow decreasing trend in perfusion while the transit-time flowmeter showed an increase. This is due to the fact that the telemetry sensor is measuring tissue perfusion directly by looking at the tissue hemoglobin content while the ultrasonic transit-time flowmeter is measuring vascular flow changes. This discrepancy between the two can be due to a systemic response causing vasoconstriction that results in a decrease in microvasculature perfusion while increasing the blood flow in the central vasculature. Such a response can be triggered by a decrease in blood pressure. To verify this theory, the change in the Mean Arterial Pressure (MAP) was looked at as shown in FIG. 11B. During the period of increase in the Transit-Time flowmeter signal, the MAP increased from 52 mmHg to more than 70 mmHg. It is believed that this event was triggered by the first portal venous occlusion that caused a decrease in venous return to the heart thereby causing a decrease in blood pressure to around 30 mmHg. This decrease in pressure is not seen during the first four occlusion events (HA occlusions) since the HA flow (350 mL/min in humans) is much lower than the PV flow (1100 mL/min, ˜22% of total cardiac output). In general, vasoconstriction is associated with an increase in MAP which supports the proposed explanation. In addition, the telemetry sensor data from the hepatic artery probe was looked at, and they showed a similar increasing trend as measured by the Transit-Time flowmeter. This is an advantage of the employed technique since perfusion measurements are desired. Flow readings are usually used as an estimate of perfusion trends; however, in addition to perfusion, these measurements are affected by changes in blood pressure. Spectroscopy based techniques measure the real hemoglobin content in tissue which is essential to know the availability of nutrients and oxygen to cells.

To assess the accuracy of the perfusion measurements, the readings from the telemetry sensor were compared with the Transit-Time flowmeter prior to the increase in blood pressure (first 4 occlusions). The telemetry sensor was able to resolve perfusion changes with an RMSE of 0.135 mL/min/g of tissue (70.87 mL/min) as shown in FIGS. 12A and 9B. Note that the standard deviation of the Transit-Time flowmeter measurements (0.09 mL/min/g of tissue [symbol] 47.6 mL/min) during the first baseline collection (t=148-155 min) is on the same order as the RMSE of the telemetry sensor.

As described above, a method for determining a venous oxygenation and an arterial oxygenation of a tissue or an organ has been disclosed. A probe is provided that is affixed to or in close proximity to a surface of the tissue or organ, or subcutaneously inserted into the tissue or organ. The probe includes one or more light sources and one or more photodetectors. The light emitted by the one or more light source may include three or more wavelengths of light (e.g., a first wavelength of approximately 735 nm, a second wavelength of approximately 805 nm, and a third wavelength of approximately 940 nm).

One or more processors communicably coupled to the probe and a data output device are also provided. The tissue or organ is illuminated using the one or more light sources, and a reflectance signal is detected using the one or more photodetectors. The reflectance signal includes a having an AC component and a DC component. The venous oxygenation and the arterial oxygenation for the tissue or organ are determined based on the reflectance signal using the one or more processors. The venous oxygenation and the arterial oxygenation for the tissue or organ are then provided to the output device.

The step of determining the venous oxygenation and the arterial oxygenation for the tissue or organ based on the reflectance signal using the one or more processors may include determining the venous oxygenation based on both the AC component the DC component of the reflectance signal, and determining the arterial oxygenation for the tissue or organ is based on the AC component of the reflectance signal. The one or more processors can further determine the arterial oxygenation and the venous oxygenation for the tissue or organ using a lookup table comprising a conversion chart wherein the value of R is described in terms of oxygen saturation for arterial blood flow, and the values obtained from Equation 2 are described in terms of oxygen saturation for venous blood flow. Moreover, a perfusion index (PI) and a heart rate (HR) from the AC component of the reflectance signal, and a change in total hemoglobin concentration (ΔHbT) from the DC component of the reflectance signal can be determined using the one or more processors. Finally, a calorie burn, an energy expenditure or a tissue oxidative stress based on a difference between the arterial oxygenation and the venous oxygenation for the tissue or organ and one or more of the perfusion index (PI), the heart rate (HR), and the change in total hemoglobin concentration (ΔHbT) can be determined.

The method may also include the step of time multiplexing or frequency multiplexing the one or more photodetectors to collect the reflectance signal at each of the three or more wavelengths of light using frequency modulation, time division multiplexing or a combination thereof. Similarly, the method may include the step of modulating the one or more light sources such that the light is at a different frequency than an ambient light.

Note that embodiments of the present invention can be used for “Non-Invasive Monitoring of Tissue Mechanical Properties” as disclosed in a non-provisional patent application filed concurrently herewith and provisional patent application Ser. No. 61/932,575 filed on Jan. 28, 2014, both having that title and incorporated by reference in their entirety.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or rives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

Although the present invention and its advantages have been described in detail, it should be understood that various changes, substitutions and alterations may be made herein without departing from the spirit and scope of the invention as defined by the appended claims. The scope of this disclosure is not limited to the example embodiments described or illustrated herein. Moreover, although this disclosure describes and illustrates respective embodiments herein as including particular components, elements, functions, operations, or steps, any of these embodiments may include any combination or permutation of any of the components, elements, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative.

What is claimed is:

1. An oxygen consumption monitoring system comprising:
a probe comprising a light source and a photodetector; and
a main unit comprising a microcontroller and a communications interface with the probe;
wherein the photodetector is configured to collect a reflectance data from a light emitted by the light source that illuminates a tissue or organ; and
wherein the microcontroller processes the reflectance data into an arterial blood oxygen consumption data and a venous blood oxygen consumption data for the tissue or organ.

2. The system as recited in claim 1, wherein the probe is hermetically sealed and is configured to be affixed to or in close proximity to a surface of the tissue or organ, or subcutaneously inserted into the tissue or organ.

3. The system as recited in claim 1, wherein the probe is integrated into, directly connected, tethered or wirelessly connected to the main unit.

4. The system as recited in claim 1, wherein the reflectance data comprises a reflectance signal having an AC component and a DC component.

5. The system as recited in claim 4, wherein the microcontroller determines the arterial blood oxygen consumption data for the tissue or organ based on the AC component of the reflectance signal, and the venous blood oxygen consumption data based on both the AC component the DC component of the reflectance signal.

6. The system as recited in claim 5, wherein the microprocessor determines the arterial blood oxygen consumption data and the venous blood oxygen consumption data for the tissue or organ using a lookup table comprising a conversion chart wherein the value of R is described in terms of oxygen saturation for arterial blood flow, and the values obtained from Equation 2 are described in terms of oxygen saturation for venous blood flow.

7. The system as recited in claim 4, wherein the microcontroller determines a perfusion index (PI) and a heart rate (HR) from the AC component of the reflectance signal, and a change in total hemoglobin concentration (ΔHbT) from the DC component of the reflectance signal.

8. The system as recited in claim 7, wherein the microcontroller determines a calorie burn, an energy expenditure or a tissue oxidative stress based on a difference between the arterial blood oxygen consumption data and the venous blood oxygen consumption data for the tissue or organ and one or more of the perfusion index (PI), the heart rate (HR), and the change in total hemoglobin concentration (ΔHbT).

9. The system as recited in claim 1, wherein the light emitted by the light source comprises three or more wavelengths of light.

10. The system as recited in claim 9, wherein the three or more wavelengths of light comprise a first wavelength of approximately 735 nm, a second wavelength of approximately 805 nm, and a third wavelength of approximately 940 nm.

11. The system as recited in claim 10, wherein the photodetector is time multiplexed or frequency multiplexed to collect the reflectance data at each of the three or more wavelengths of light using frequency modulation, time division multiplexing or a combination thereof.

12. The system as recited in claim 1, wherein the light source modulated the light such that the light is at a different frequency than an ambient light.

13. A method for determining a venous oxygenation and an arterial oxygenation of a tissue or an organ, comprising the steps of:
providing a probe affixed to or in close proximity to a surface of the tissue or organ, or subcutaneously inserted into the tissue or organ, wherein the probe comprises one or more light sources and one or more photodetectors;
providing one or more processors communicably coupled to the probe and a data output device;
illuminating the tissue or organ using the one or more light sources;
detecting a reflectance signal using the one or more photodetectors;
determining the venous oxygenation and the arterial oxygenation for the tissue or organ based on the reflectance signal using the one or more processors; and
providing the venous oxygenation and the arterial oxygenation for the tissue or organ to the output device.

14. The method as recited in claim 13, wherein the reflectance signal comprises an AC component and a DC component.

15. The method as recited in claim 14, wherein the step of determining the venous oxygenation and the arterial oxygenation for the tissue or organ based on the reflectance signal using the one or more processors comprises determining the venous oxygenation based on both the AC component the DC component of the reflectance signal, and determining the arterial oxygenation for the tissue or organ is based on the AC component of the reflectance signal.

16. The method as recited in claim 15, wherein the one or more processors further determine the arterial oxygenation and the venous oxygenation for the tissue or organ using a lookup table comprising a conversion chart wherein the value of R is described in terms of oxygen saturation for arterial blood flow, and the values obtained from Equation 2 are described in terms of oxygen saturation for venous blood flow.

17. The method as recited in claim 14, further comprising the step of determining a perfusion index (PI) and a heart rate (HR) from the AC component of the reflectance signal, and a change in total hemoglobin concentration (ΔHbT) from the DC component of the reflectance signal using the one or more processors.

18. The method as recited in claim 17, further comprising the step of determining a calorie burn, an energy expenditure or a tissue oxidative stress based on a difference between the arterial oxygenation and the venous oxygenation for the tissue or organ and one or more of the perfusion index (PI), the heart rate (HR), and the change in total hemoglobin concentration (ΔHbT).

19. The method as recited in claim 13, wherein the light emitted by the one or more light source comprises three or more wavelengths of light.

20. The method as recited in claim 19, wherein the three or more wavelengths of light comprise a first wavelength of approximately 735 nm, a second wavelength of approximately 805 nm, and a third wavelength of approximately 940 nm.

21. The method as recited in claim 20, further comprising the step of time multiplexing or frequency multiplexing the one or more photodetectors to collect the reflectance signal at each of the three or more wavelengths of light using frequency modulation, time division multiplexing or a combination thereof.

22. The method as recited in claim 13, further comprising the step of modulating the one or more light sources such that the light is at a different frequency than an ambient light.
